(19) United States
(12) Patent Application Publication
(10) Pub. No.: US 2024/0161937 A1
(43) Pub. Date: May 16, 2024

(54) METHOD AND APPARATUS FOR PROVIDING CLOUD-BASED MEDICAL COLLABORATION SERVICE FOR MEDICAL IMAGES

(71) Applicant: Promedius Inc., Seoul (KR)

(72) Inventors: Hyun Jin BAE, Seoul (KR); Min Gyu KIM, Seoul (KR)

(73) Assignee: Promedius Inc., Seoul (KR)

(21) Appl. No.: 18/156,509

(22) Filed: Jan. 19, 2023

(30) Foreign Application Priority Data
Nov. 11, 2022 (KR) 10-2022-0150247

(51) Int. Cl. G16H 80/00; G06F 21/62; G16H 30/20

(52) U.S. Cl. CPC G16H 80/00; G06F 21/6254; G16H 30/20

(57) ABSTRACT

Provided are a method and an apparatus for providing a cloud-based medical collaboration service for medical images. Even if a plurality of users are not in the same space at the same time, since they can provide their opinions at desired time and place through data storing and sharing, remote collaboration is possible without restrictions for physical time and place. Moreover, an AI solution-based artificial intelligence model makes remote collaboration among a plurality of medical experts possible.

### CROSS-REFERENCE TO RELATED APPLICATION

A claim for priority under 35 U.S.C. § 119 is made to Korean Patent Application No. 10-2022-0150247 filed on Nov. 11, 2022 in the Korean Intellectual Property Office, the entire contents of which are hereby incorporated by reference.

### BACKGROUND

### 1. Technical Field

The present disclosure relates to a method and an apparatus for providing a cloud-based medical collaboration service for medical images.

### 2. Description of Related Art

A medical practice refers to an action to prevent or treat diseases by performing diagnosis, optometry, prescription, medication, or surgical procedures based on medical expertise.

In general, a medical team diagnoses a patient's condition alone when performing medical treatment. This method may lower accuracy of diagnosis in a case in which the patient's condition is complicated or the medical team requires the advice of experts in different fields. Accordingly, a platform for medical collaboration among various experts is required.

### SUMMARY

In an aspect of the present disclosure, an object of the present disclosure is to provide a method and an apparatus for providing a cloud-based medical collaboration service for medical images.

The aspects of the present disclosure are not limited to those mentioned above, and other aspects not mentioned herein will be clearly understood by those skilled in the art from the following description.

To accomplish the above-mentioned objects, according to an aspect of the present disclosure, there is provided a method for providing a cloud-based medical collaboration service for medical images including operations of: acquiring original data including medical images from a user terminal; deidentifying personal identification information included in the original data; mapping and storing the original data in which the personal identification information is deidentified (deidentified data) and the personal identification information; providing the deidentified data to at least one further user terminal; obtaining comment information on the deidentified data from the at least one further user terminal; and further mapping and storing the comment information to the mapped deidentified data and personal identification information.

Moreover, in the further mapping and storing the comment information, the comment information is arranged and stored based on relevance between a medical field of the medical images and a medical department of at least one further user, and the career period of the at least one further user.

Furthermore, the method further includes an operation of: providing result data to the user terminal, wherein the result data includes the personal identification information and the comment information mapped to the deidentified data.

Additionally, the user terminal is linked to a storage of the service platform through an API.

In addition, the method further includes an operation of: constructing an artificial intelligence model using medical data stored in terminals of all users joining the service platform.

In addition, the method further includes the operation of: calculating a score based on accuracy of a prediction result of the artificial intelligence model; and updating the artificial intelligence model using the prediction result, wherein the updating is performed by reflecting a weighted value based on the calculated score.

Moreover, in a case in which manipulation of the original data is performed by the user terminal in a state in which the deidentified data is provided to another user terminal, the manipulation is reflected in the deidentified data displayed on the terminal of the another user in the same manner.

Furthermore, the user terminal and the at least one further user terminal perform labeling of the medical images in real time using a labeling tool, and the deidentified images and the original data for the labeling work are displayed on separate windows of the terminal of the user.

To accomplish the above-mentioned objects, according to another aspect of the present disclosure, there is provided an apparatus for providing a cloud-based medical collaboration service for medical images, including: a communication unit; a memory storing at least one process for providing a cloud-based medical collaboration service for medical images; and a processor operating according to the process, wherein the processor acquires original data including medical images from a user terminal, deidentifies personal identification information included in the original data, maps and stores the original data in which the personal identification information is deidentified (deidentified data) and the personal identification information, provides the deidentified data to at least one further user terminal, obtains comment information on the deidentified data from the at least one further user terminal, and further maps the comment information to the mapped deidentified data and personal identification information and stores the comment information in the memory.

Besides the above, a computer program stored in a computer readable recording medium for embodying the present disclosure may be additionally provided.

Besides the above, a computer readable recording medium to record computer programs for executing the method may be additionally provided.

### BRIEF DESCRIPTION OF THE DRAWINGS

FIG. 1 is a schematic diagram illustrating a system for providing a cloud-based medical collaboration service for medical images according to the present disclosure.

FIG. 2 is a schematic diagram illustrating an apparatus for providing a cloud-based medical collaboration service for medical images according to the present disclosure.

FIG. 3 is a flow chart illustrating a method for providing a cloud-based medical collaboration service for medical images according to the present disclosure.

FIG. 4 is a diagram for depicting original data and deidentified data according to the present disclosure.

FIG. 5 is a diagram for depicting result data according to the present disclosure.

### DETAILED DESCRIPTION

In the drawings, like reference numerals designate like components. This disclosure does not describe all components of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one component, or a unit, a module, a member, or a block may include a plurality of components.

Throughout this specification, when a part is referred to as being “connected” to another part, this includes “direct connection” and “indirect connection,” and the indirect connection may include connection via a wireless communication network.

Furthermore, when a certain part “includes” a certain component, other components are not excluded unless explicitly described otherwise, and other components may in fact be included.

In the entire specification of the present disclosure, when any member is located “on” another member, this includes a case in which still another member is present between both members as well as a case in which one member is in contact with another member.

The terms “first,” “second,” and the like are just to distinguish a component from any other component, and components are not limited by the terms.

The singular form of the components may be understood into the plural form unless otherwise specifically stated in the context.

Identification codes in each operation are used not for describing the order of the operations but for convenience of description, and the operations may be implemented differently from the order described unless there is a specific order explicitly described in the context.

Hereinafter, operation principles and embodiments of the present disclosure will be described with reference to the accompanying drawings.

In this specification, an apparatus includes various devices capable of performing arithmetic processing to provide results to a user. For example, the apparatus 100 according to an embodiment of the present disclosure may include all of a computer, a server device, and a portable terminal, or may adopt any one thereamong.

Here, the computer may include, for example, a notebook computer equipped with a web browser, a desktop, a laptop, a tablet PC, a slate PC, and the like.

The server device is a server for processing information by performing communication with the external device, and includes an application server, a computing server, a database server, a file server, a game server, a mail server, a proxy server, a web server, and the like.

The portable terminal is a wireless communication device providing portability and mobility, and includes all kinds of handheld-based wireless communication devices, such as a personal communications system (PCS), a global system for mobile communications (GSM), a personal digital cellular (PDC), a personal handphone system (PHS), a personal digital assistant (PDA), an international mobile telecommunications (IMT)-2000, a code division multiple access (CDMA)-2000, a W-code division multiple access (W-CDMA), wireless broadband internet (WiBro) terminal, a smartphone, and the like, and a wearable device, such as a watch, a ring, a bracelet, an ankle bracelet, a necklace, glasses, contact lenses, or a head-mounted device (HMD).

In this specification, ‘medical collaboration’ may be interpreted as meaning including both remote collaboration and remote interpretation. That is, even if medical teams cooperate with each other with respect to medical images of a patient, the medical collaboration may be performed according to the process of the present disclosure. In the same manner, also in a case in which a medical doctor shares medical images of the doctor's patient and another medical doctor interprets the corresponding medical images, the medical interpretation may be performed according to the process of the present disclosure.

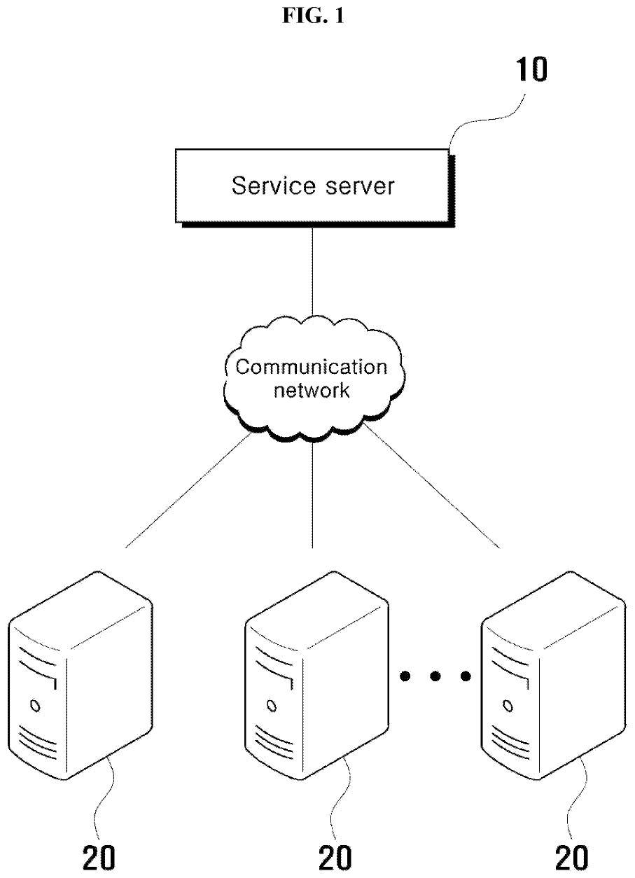

FIG. 1 is a schematic diagram illustrating a system for providing a cloud-based medical collaboration service for medical images according to the present disclosure.

Referring to FIG. 1, the system according to the present disclosure may include a service server 10 and a user terminal 20. However, in some embodiments, the system may include fewer or more components than those illustrated in FIG. 1.

The service server 10 is a cloud-based server, and allows all users who have joined the service to share data uploaded by each other through a cloud computing service.

The user terminal 20 is a device used by a medical professional (e.g., a doctor) and may mean a server or an external PC equipped with a graphic processing unit (GPU) possessed by the user.

The user terminal 20 may be applied with an information processing means such as a computer, may include a processor such as a controller, a photographing means such as a camera, and an input/output means including a touch screen, and may refer to any device including a communication function. That is, the user terminal may be any device such as a smartphone, a tablet, a PDA, a laptop PC, or a desktop PC.

In a case in which a user uploads original data including medical images to the cloud through the user terminal 20 (or synchronizes original data from the user's local folder linked to the cloud), the system of the present disclosure does not share the original data with another user as it is but may perform deidentification process for personal identification information contained in the original data and share deidentified data with the another user.

Thereafter, in a case in which the user downloads the data that comments from the another user have been input to a local folder, or the like, personal identification information is contained into the deidentified data so that the user can confirm all of personal identification information, medical images, and comment information.

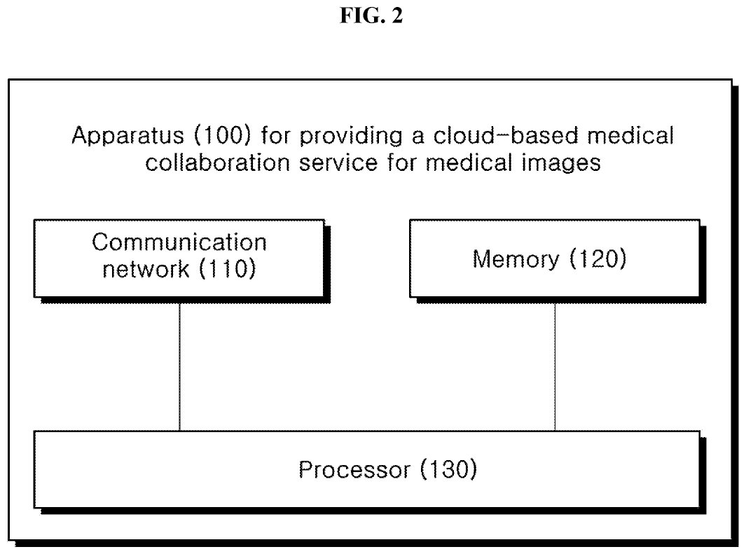

FIG. 2 is a schematic diagram illustrating an apparatus for providing a cloud-based medical collaboration service for medical images according to the present disclosure.

Referring to FIG. 2, the apparatus 100 for providing a cloud-based medical collaboration service for medical images according to the present disclosure (hereinafter, a service providing apparatus) may include a communication unit 110, a memory 120 and a processor 130. However, in some embodiments, the service providing apparatus 100 may include fewer or more components than those illustrated in FIG. 2. The service providing apparatus 100 to be described with reference to FIG. 2 may be the service server 10 described with reference to FIG. 1.

The communication unit 110 may include one or more components enabling communication with the user terminal 20 or an external device (not illustrated), and may include at least one among, for example, a wired communication module, a wireless communication module, and a short-distance communication module, and a location information module.

The wired communication module may include not only various wired communication modules, such as a local area network (LAN) module, a wide area network (WAN) module, or a value added network (VAN) module, but also various cable communication modules, such as a universal serial bus (USB), a high definition multimedia interface (HDMI), a digital visual interface (DVI), a recommended standard 232 (RS-232), a power line communication, or a plain old telephone service (POTS).

The wireless communication module may support various wireless communication methods, such as global system for mobile communication (GSM), code division multiple access (CDMA), wideband code division multiple access (WCDMA), universal mobile telecommunications system (UMTS), time division multiple access (TDMA), long term evolution (LTE), and 4G, 5G, 6G and the like as well as a Wi-Fi module, a wireless broadband (Wibro) module.

The short-distance communication module may support short-distance communication using at least one among Bluetooth™, radio frequency identification (RFID), infrared data association (IrDA), ultra-wideband (UWB), ZigBee®, near field communication (NFC), wireless-fidelity (Wi-Fi), Wi-Fi Direct, and wireless universal serial bus (Wireless USB).

The memory may store at least one process for providing a cloud-based medical collaboration service for medical images.

The memory 120 may store data supporting various functions of the service providing apparatus 100, and a program for operating the processor 130, may store input/output data (e.g., music files, still images, moving images, etc.), and may store a plurality of application programs (application programs or applications) running on the apparatus of the present disclosure, and data and instructions for operating the apparatus 100. At least a portion of these applications may be downloaded from an external server via wireless communication.

The memory 120 may include a storage medium having at least one among a flash memory type memory, a hard disk type memory, a multimedia card micro type memory, a card type memory (e.g., an SD memory or an XD memory), a random access memory (RAM), static random access memory (SRAM), read-only memory (ROM), an electrically erasable programmable read-only memory (EEPROM), a programmable read-only memory (PROM), a magnetic memory, a magnetic disk, and an optical disk. Furthermore, although the memory unit is separated from the apparatus, it may be a database connected by wire or wirelessly.

The processor 130 may perform prescribed operations using the memory storing algorithm for controlling operations of the components of the apparatus 100 or data of programs reproducing the algorithms, and data stored in the memory. In this instance, the memory 120 and the processor 130 may be implemented as separate chips. Alternatively, the memory 120 and the processor 130 may be implemented as a single chip.

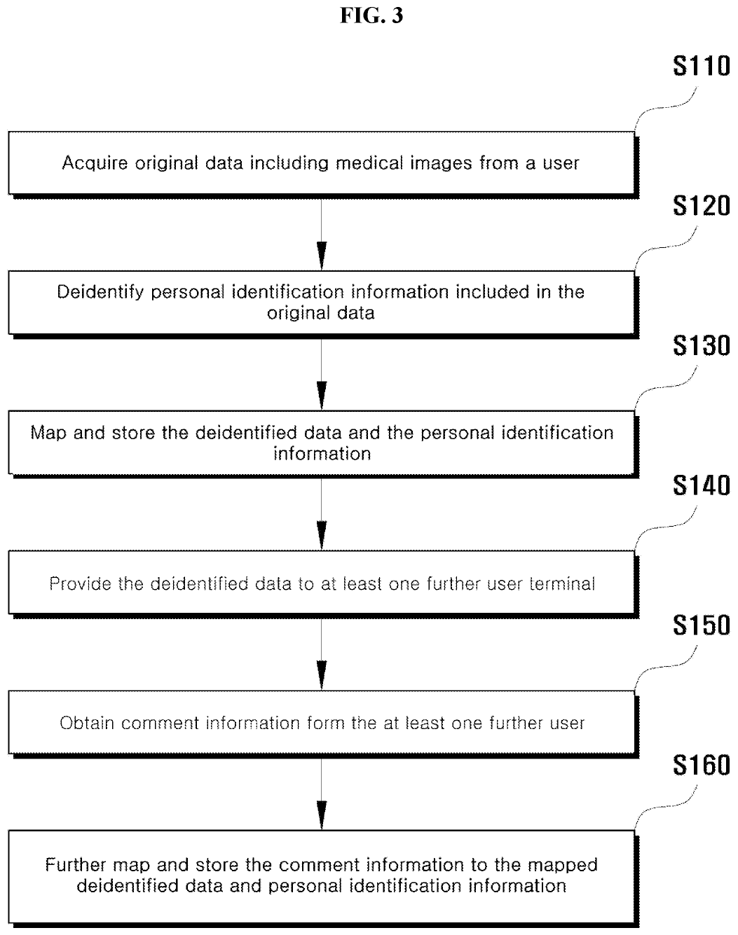

In addition, the processor 130 may control by combining any one or the plurality of components in order to apply various embodiments according to the present disclosure described in FIGS. 3 to 5 to the service providing apparatus 100.

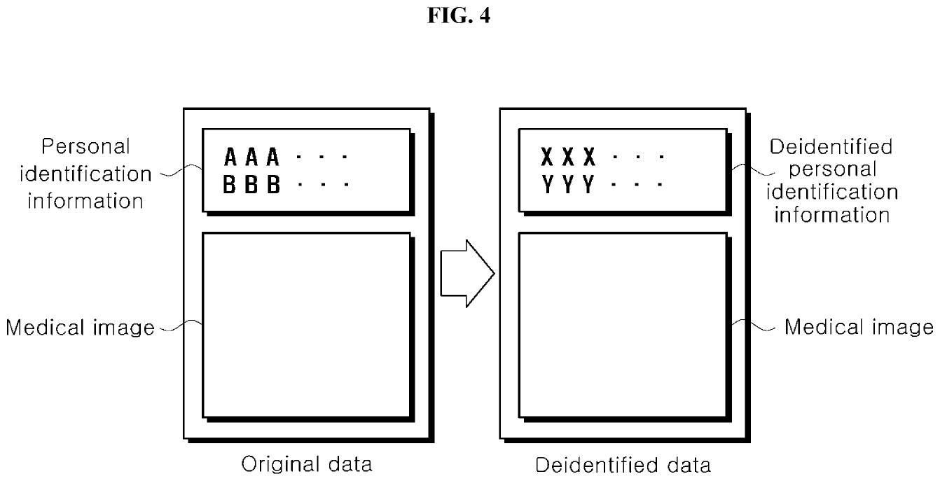

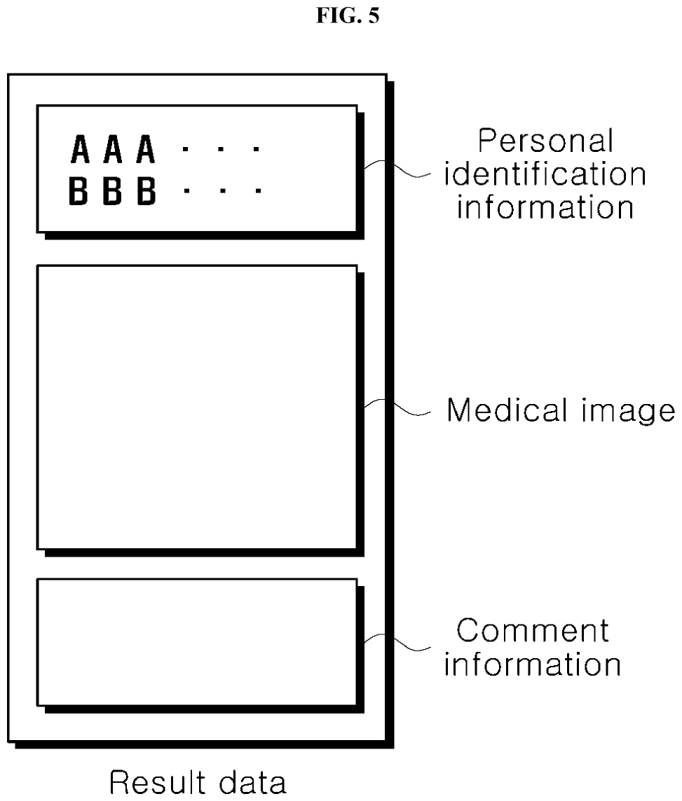

FIG. 3 is a flow chart illustrating a method for providing a cloud-based medical collaboration service for medical images according to the present disclosure. FIG. 4 is a diagram for depicting original data and deidentified data according to the present disclosure. FIG. 5 is a diagram for depicting result data according to the present disclosure. Hereinafter, the method of FIG. 3 will be described as being performed by the service providing apparatus 100, but is not limited thereto, and it may also be understood that the method of FIG. 3 is performed by the service server 10 of FIG. 1.

Referring to FIG. 3, the processor 130 of the service providing apparatus 100 may obtain original data including medical images from the user terminal 20 (S110).

The processor 130 of the service providing device 100 may de-identify the personal identification information included in the original data (S120).

The processor 130 of the service providing device 100 may map and store the original data in which personal identification information is deidentified (hereinafter, deidentified data) and the personal identification information (S130).

The processor 130 of the service providing apparatus 100 may provide the deidentified data to at least one other user terminal 20 (S140).

The processor 130 of the service providing apparatus 100 may obtain comment information on the deidentified data from the terminal 20 of the another user (S150).

The processor 130 of the service providing device 100 may further map and store comment information on the mapped deidentified data and the personal identification information (S160).

Hereinafter, the operation S110 will be described in detail.

The processor 130 may obtain original data uploaded to the cloud through the user terminal 20 by a user (medical expert) having requested medical collaboration.

Alternatively, the processor 130 may acquire the original data that the user (medical expert) requesting collaboration put in a cloud-linked local folder in the user terminal 20 through synchronization.

Here, the original data may include personal identification information for identifying a patient, and medical images for determining the patient's condition, but is not limited thereto, and may include various types of information for diagnosis. Medical images may refer to image data captured by various methods, such as CT, MRI, angiography, and ultrasound as well as X-rays.

In addition, the user terminal 20 may be linked to a storage of the service platform through an application programming interface (API). Therethrough, the image (data) stored in the user terminal 20 can be sent to the service platform to be stored and utilized. On the contrary, the user terminal 20 may retrieve the image (data) stored in the service platform and use it for learning or reasoning.

Next, the operations S120 and S130 will be described in detail.

After deidentifying the personal identification information included in the acquired original data, the processor 130 may map the original data in which the personal identification information is deidentified (hereinafter, deidentified data) and the personal identification information, and then, store the deidentified original data and the personal identification information in the memory 120.

Here, the de-identification process includes pseudonymization that replaces all or a portion of data with other values, deletion that erases all or a portion of data, categorization that hides exact values of data and converts data into category values, and data masking that makes important identifiers invisible.

Referring to FIG. 4, in a case in which the original data including personal identification information (e.g., AAA, BBB) and medical images are deidentified, the personal identification information is deidentified (e.g., XXX, YYY) and the medical images are maintained as they are in the de-identification data.

Next, the operations S140 and S150 will be described in detail.

The processor 130 may provide deidentified original data to another user for collaboration. That is, as illustrated in FIG. 4, data in which the personal identification information is deidentified and only the medical images are identifiable are provided to another user terminal 20, so that other users cannot confirm information on the patient and can give a comment while watching only the medical images.

As described above, other users can provide a more objective diagnosis since judging the patient's condition by watching only the medical images without the personal information about the patient.

Here, the another user may mean a medical expert different from the user requesting collaboration. In the present disclosure, the user and other users may refer to people who join the service platform to use the medical collaboration service. That is, the another user may also request medical collaboration, and the user may also give a comment about data provided by the another user so that the user and the another user may give and receive help each other.

Next, the operation S160 will be described in detail.

The processor 130 may further map and store the comment information acquired from the another user to the deidentified data and the personal identification information previously mapped and stored in the memory 120. That is, deidentified data, personal identification information, and comment information may be mapped and stored together in the memory 120.

As described above, since the original data shared by the user are not stored as they are but deidentified data and personal identification information separated from the original data are mapped and stored, comment information received from the another user is also mapped and stored together with the previously mapped data.

In this instance, in a case in which at least one piece of comment information is obtained from at least one other user terminal 20, the processor 130 may arrange and store at least one piece of comment information based on relevance between the medical field of the medical images and the medical department of at least one further user, and the career period of the at least one further user.

Here, the medical department in charge and the career period may be information input by the users (the user and other users) when joining the service platform.

In addition, according to embodiments, the medical field of the medical images may be information input by the user when the user requesting collaboration shares the original data.

According to embodiments, the medical field of the medical images may be determined by the processor 130 based on the medical department of the user requesting collaboration.

According to embodiments, the processor 130 may determine the medical field of the medical images based on information included in the original data.

According to embodiments, the medical field of the medical images may be determined by the processor 130 analyzing the medical images included in the original data.

In a case in which a plurality of comment information is acquired from a plurality of different user terminals 20, the plurality of comment information may be arranged and stored in order according to a predetermined criterion.

According to embodiments, the processor 130 may calculate the degree of relevance between the medical field of the medical images and the medical department of at least one further user, and prioritize and arrange comment information of the another user having a high degree of relevance. For example, if the medical field of the medical images and the medical department of at least one further user are the same, the relevance is the highest. In a case in which the medical field of the medical images is neurology and the medical department of the another user is neurosurgery, the relevance is high because they are highly related.

According to embodiments, the processor 130 compares the career periods of other users, and prioritizes and arrange comment information of the another user having a long career period.

According to embodiments, the processor 130 may prioritize and arrange comment information of the another user whose medical department has a high degree of relevance to the medical field of medical images and who has a long career period. Specifically, a first weighted value and a second weighted value are respectively applied to the degree of relevance and the career period, first weighted values of different values are applied according to the calculated relevance values for other users, and second weighted values of different values are respectively applied according to career periods of other users. As described above, weighted values of different values are applied according to the degree of relevance and the career periods to calculate total score, and the comment information may be sorted in order of higher total scores.

Thereafter, the processor 130 may provide result data to the user terminal 20.

Here, the result data may include the comment information and the personal identification information mapped with respect to the deidentified data.

That is, in a case in which comments are input from other users with respect to the deidentified data (more precisely, medical images included in the deidentified data), the user requesting collaboration may download the result data into a local folder in the user terminal 20 through the cloud to confirm the result data.

As illustrated in FIG. 5, the user can confirm the result data. That is, although deidentified data (e.g., XXX, YYY), personal identification information (e.g., AAA, BBB), and comment information are mapped and stored in the memory 120 as individual data, deidentified data, personal identification information, and comment information may be provided to the user's local folder as a piece of data (result data) based on the mapping information. More specifically, according to the mapping information, a deidentified portion of the deidentified data may be identified again and comment information may be added to be provided to the user as a piece of data.

According to an embodiment of the present disclosure, in a state in which the deidentified data is provided to another user's terminal 20, in a case in which manipulation of the original data is performed in the user terminal 20, the manipulation may be equally reflected to the deidentified data displayed on the terminal 20 of the another user.

Here, the manipulation may include slice movement, window change, and rendering. Specifically, in a case in which the user terminal 20 includes a touch display, when the user moves two fingers on a screen, slice movement may be performed on 3D images such as CT or MRI. The width of a window value is changed when the user moves one hand on the screen laterally, and the level of the window may be changed when the user moves one hand vertically.

That is, in a case in which the user manipulates the medical images or the personal identification information in the original data through the user terminal, the corresponding manipulation may be reflected and displayed on deidentified data displayed on the terminal of the another user. However, in this instance, manipulation of medical images is equally reflected, but manipulation of the personal identification information may not be reflected on the screen of the terminal of the another user (because the personal identification information is deidentified in deidentified data).

According to an embodiment of the present disclosure, the user terminal 20 and the at least one further user terminal 20 may perform labeling of the medical images in real time using a labeling tool. That is, the user and the another user can access the platform at the same time and discuss the labeling work together while watching the patient's medical images through each terminal.

In this instance, since the original data and the deidentified data may be displayed on the user's terminal 20 and the at least one further user terminal 20, the processor 130 allows the deidentified images for labeling work and the original data to be respectively displayed on separate windows of the user terminals 20, so that the user and the another user can perform work while watching the same data. Therefore, the user and the another user can perform work while watching the same deidentified data (namely, medical images).

Alternatively, according to an embodiment, the original data and the deidentified data are displayed on the user terminal 20 and the at least one further user terminal 20, so that the user and the another user perform labeling work. In a case in which the user performs labeling work for the personal identification information in the original data, the deidentification data displayed on the terminal 20 of the another user may not display a result of the corresponding work.

According to an embodiment of the present disclosure, the processor 130 may reduce the loading speed when users upload data to the service platform using a pre-built compression algorithm.

According to an embodiment of the present disclosure, the processor 130 may obtain comment information from another user and a report prepared by the another user. In this instance, in order to distinguish authenticity of the report prepared by the another user in a remote collaboration process, a hash key may be stored in a private or public blockchain. Thereafter, in a case in which the user browses the corresponding report, verification can be performed through the corresponding hash key information based on a smart contract.

In addition, not illustrated in FIG. 3, but the method for providing a cloud-based medical collaboration service for medical images according to the present disclosure may further include the operation of constructing an artificial intelligence model using medical-related data stored in terminals 20 of all users joining the service platform.

As described above, the processor 130 may retrieve and utilize the data stored in the terminals 20 of all users joining the service through API interworking.

In this instance, the medical-related data may include medical image data, diagnosis data, and treatment data according to patients, ages, genders, smoking, and drinking, and also include the result data (data including all of personal identification information of a specific patient, medical images, and comment information). The processor 130 may construct an artificial intelligence model by learning various medical data.

In a case in which users store the medical-related data in the users' terminals 20 to construct an artificial intelligence model, the processor 130 may retrieve the medical-related data stored in each specific folder and utilize it as learning data of the artificial intelligence model.

According to an embodiment, the processor 130 may calculate the level of contribution of each user based on a ratio of the total quantity of learning data used to construct the artificial intelligence model and the quantity of data provided by the users, and distribute data to the users according to the calculated contribution.

When the artificial intelligence model is constructed, the processor 130 may perform prediction on the medical image input by the user. The user can diagnose the patient's condition based on the results predicted by the artificial intelligence model.

In addition, the processor 130 may calculate a score based on accuracy of the prediction result of the artificial intelligence model, and update the artificial intelligence model using the prediction result. In this instance, the processor 130 may update the artificial intelligence model by reflecting a weighted value based on the calculated score.

Here, the accuracy of the prediction result may be determined based on the feedback of the user who input the corresponding medical images. Specifically, it can be understood that the accuracy is high as the diagnosis result of the user is similar to the prediction result.

Thereafter, the artificial intelligence model may be updated using prediction results for various medical images. In this instance, the medical images having low scores in accuracy and the corresponding prediction result may be learned with low weighted values, and the medical images having high scores in accuracy and the corresponding prediction result may be learned with high weighted values.

According to an embodiment of the present disclosure, the processor 130 may use the artificial intelligence model loaded in the service platform alone or in combination (=pipeline). For example, image data of the chest may be input to a model A to output result data, and then, the output result data may be input to a model B to be analyzed.

To date, it has been described that the artificial intelligence model learns a medical-related data uploaded to the service platform to be constructed, but the present disclosure is not limited thereto, and an artificial intelligence model constructed not on the service platform but outside the service platform may be used. In addition, depending on embodiments, an artificial intelligence model constructed outside may be combined with the artificial intelligence model constructed through learning of the medical-related data in the service platform to be utilized.

Although FIG. 3 illustrates operations being sequentially executed, it is only to exemplify the technical idea of this embodiment. Accordingly, it will be understood by the skilled persons in the art that the sequence of operations illustrated in FIG. 3 may be changed, or one or more operations illustrated in FIG. 3 may be executed in parallel without deviating from the essential characteristics of the present embodiment. That is, since various modifications and variations may be applied to this embodiment, FIG. 3 is not limited to the time sequential order.

Meanwhile, in the above description, the operations described in FIG. 3 may be further divided into additional operations or combined into fewer operations according to an embodiment of the present disclosure. Furthermore, some operations may be omitted if necessary, and the order of operations may be changed.

According to the present disclosure, by referring to comments of other users, the user can diagnose the patient's condition with higher accuracy, thereby providing high-quality medical services to the user.

Additionally, in a case in which data including patient's medical images are provided to other users, the patient's personal identification information is provided in a deidentified state to prevent the patient's personal information from being released.

In addition, even if a plurality of users are not in the same space at the same time, since they can provide their opinions at desired time and place, remote collaboration is possible without restrictions for physical time and place.

On the other hand, the disclosed embodiments may be implemented in the form of a recording medium storing instructions executable by a computer. Instructions may be stored in the form of program code and, when executed by a processor, may generate a program module to perform the operation of the disclosed embodiments. The recording medium may be embodied as a computer-readable recording medium.

The computer readable recording medium includes all kinds of recording media in which instructions that can be decrypted by a computer are stored. For example, there may be a read-only memory (ROM), a random access memory (RAM), a magnetic tape, a magnetic disk, a flash memory, an optical data storage device, and the like.

The above description is only exemplary, and it will be understood by those skilled in the art that the disclosure may be embodied in other concrete forms without changing the technological scope and essential features. Therefore, the above-described embodiments should be considered only as examples in all aspects and not for purposes of limitation.

1. A method for providing a cloud-based medical collaboration service for medical images, which is performed by an apparatus, the method comprising the operations of:
acquiring original data including medical images from a user terminal;
deidentifying personal identification information included in the original data;
mapping and storing the original data in which the personal identification information is deidentified (deidentified data) and the personal identification information;
providing the deidentified data to at least one further user terminal;
obtaining comment information on the deidentified data from the at least one further user terminal; and
further mapping and storing the comment information to the mapped deidentified data and personal identification information.

2. The method according to claim 1, wherein in the further mapping and storing the comment information, the comment information is arranged and stored based on relevance between a medical field of the medical images and a medical department of at least one further user, and the career period of the at least one further user.

3. The method according to claim 1, further comprising the operation of:
providing result data to the user terminal,
wherein the result data includes the personal identification information and the comment information mapped to the deidentified data.

4. The method according to claim 1, wherein the user terminal is linked to a storage of the service platform through an API.

5. The method according to claim 4, further comprising the operation of:
constructing an artificial intelligence model using medical data stored in terminals of all users joining the service platform.

6. The method according to claim 5, further comprising the operation of:
calculating a score based on accuracy of a prediction result of the artificial intelligence model; and
updating the artificial intelligence model using the prediction result, wherein the updating is performed by reflecting a weighted value based on the calculated score.

7. The method according to claim 1, wherein in a case in which manipulation of the original data is performed by the user terminal in a state in which the deidentified data is provided to another user terminal, the manipulation is reflected in the deidentified data displayed on the terminal of the another user in the same manner.

8. The method according to claim 1, wherein the user terminal and the at least one further user terminal perform labeling of the medical images in real time using a labeling tool, and
wherein the deidentified images and the original data for the labeling work are displayed on separate windows of the terminal of the user.

9. An apparatus for providing a cloud-based medical collaboration service for medical images, the apparatus comprising:
a communication unit;
a memory storing at least one process for providing a cloud-based medical collaboration service for medical images; and
a processor operating according to the process,
wherein the processor:
acquires original data including medical images from a user terminal;
deidentifies personal identification information included in the original data;
maps and stores the original data in which the personal identification information is deidentified (deidentified data) and the personal identification information;
provides the deidentified data to at least one further user terminal;
obtains comment information on the deidentified data from the at least one further user terminal; and
further maps the comment information to the mapped deidentified data and personal identification information and stores the comment information in the memory.

10. The apparatus according to claim 9, wherein in order to further map and store the comment information, the processor arranges and stores the comment information based on relevance between a medical field of the medical images and a medical department of at least one further user, and the career period of the at least one further user.

11. The apparatus according to claim 9, wherein the processor provides result data to the user terminal, and
wherein the result data includes the personal identification information and the comment information mapped to the deidentified data.

12. The method according to claim 9, wherein the user terminal is linked to a storage of the service platform through an API.

13. The method according to claim 12, wherein the processor constructs an artificial intelligence model using medical data stored in terminals of all users joining the service platform.

14. The method according to claim 13, wherein the processor calculates a score based on accuracy of a prediction result of the artificial intelligence model, and updates the artificial intelligence model using the prediction result, and updating is performed by reflecting a weighted value based on the calculated score.

15. The method according to claim 9, wherein in a case in which manipulation of the original data is performed by the user terminal in a state in which the deidentified data is provided to another user terminal, the manipulation is reflected in the deidentified data displayed on the terminal of the another user in the same manner.

16. The method according to claim 9, wherein the user terminal and the at least one further user terminal perform labeling of the medical images in real time using a labeling tool, and
wherein the deidentified images and the original data for the labeling work are displayed on separate windows of the terminal of the user.
